(19) United States
(12) Patent Application Publication
(10) Pub. No.: US 2023/0241095 A1
(43) Pub. Date: Aug. 3, 2023

(54) PARTIALLY DESULFATED HEPARIN FOR TREATING CORONAVIRAL INFECTIONS

(71) Applicant: IHP Therapeutics Inc., San Carlos, CA (US)

(72) Inventors: John Eric Paderi, San Carlos, CA (US); Robert Van Gorp, Liberty Township, OH (US); Rinko Ghosh, San Francisco, CA (US)

(21) Appl. No.: 18/009,654

(22) PCT Filed: Jun. 14, 2021
(86) PCT No.: PCT/US21/37265

§ 371 Date: Dec. 9, 2022

Related U.S. Application Data
(60) Provisional application No. 63/038,472, filed on Jun. 12, 2020.
(60) Provisional application No. 63/067,702, filed on Aug. 19, 2020.

(51) Int. Cl. A61K 31/727; A61P 31/14; A61K 31/513; A61K 31/427; A61K 31/215; A61K 31/4965; A61K 31/137; A61K 31/573; A61K 31/4706; A61K 31/675; A61K 39/395

(52) U.S. Cl. CPC A61K 31/727; A61P 31/14; A61K 31/513; A61K 31/427; A61K 31/215; A61K 31/4965; A61K 31/137; A61K 31/573; A61K 31/4706; A61K 31/675; A61K 39/3955

(57) ABSTRACT

Provided are compositions and methods for treating an infection by a coronavirus in a subject in need thereof. The composition includes an effective amount of a heparin having reduced sulfation and/or reduced anticoagulation activity as compared to unfractionated porcine intestine heparin (UPIH).

### CROSS REFERENCE TO RELATED APPLICATIONS

This application claims the benefit under 35 U.S.C. § 119(e) to U.S. Provisional Application Nos. 63/038,472, filed Jun. 12, 2020, and 63/067,702, filed Aug. 19, 2020, which are hereby incorporated by reference in their entirety.

### BACKGROUND

Coronaviruses are a group of related RNA viruses which can cause respiratory tract infections in humans. Mild illnesses include some cases of the common cold, while more lethal varieties can cause SARS, MERS, and COVID-19. Symptoms in other species vary. In chickens, they cause an upper respiratory tract disease, while in cows and pigs they cause diarrhea.

The current outbreak and global spread of new respiratory virus SARS-CoV-2 is the latest and most deadly coronavirus epidemic in the last 20 years. Like its cousin SARS-CoV, SARS-CoV-2 (or 2019 nCoV for novel coronavirus 2019) belongs to the betacoronavirus genus and likely originated in bats. Like other coronaviruses, SARS CoV-2 is believed to utilize a large surface protein called spike (S) protein for interaction with and entry into the target cell. The S protein consists of an N-terminal 51 domain followed by membrane-proximal S2 domain, a transmembrane domain and an intracellular domain. The 51 domain is responsible for interacting with the target cell through a receptor binding subdomain (RBD) while the S2 domain mediates membrane fusion following receptor binding. The viral RNA genome is then released into the cytoplasm and replicates itself. New virions can be assembled and burst out of the cell to start the whole cycle of infection again.

Understanding the forgoing process is the basis for developing novel antiviral agents in general and against SARS-CoV-2 in particular. Therapeutic strategies that may be contemplated include blocking cellular adhesion and entry, RNA synthesis and viral release as well as boosting host's immune system. Since cellular binding is the very first step that triggers viral infection, in the case of coronavirus, efforts have been taken to target the spike protein in a manner that blocks its interaction with the cellular receptor thereby forestalling infection. For SARS-CoV-2, it has been shown that human angiotensin converting enzyme II (ACE2) is the membrane receptor to which the spike protein binds.

### SUMMARY

The present disclosure, in one embodiment, provides a method for treating an infection by a coronavirus in a subject in need thereof, comprising administering to the subject an effective amount of a heparin having reduced sulfation as compared to unfractionated porcine intestine heparin (UPIH).

In another embodiment, provided is a method for treating an infection by a coronavirus in a subject in need thereof, comprising administering to the subject an effective amount of a heparin having from 10% to 70% reduction of anti-factor IIa (Ma) or anti-factor Xa (fXa) activity as compared to unfractionated porcine intestine heparin (UPIH). In certain embodiments, the heparin is a bovine heparin.

In another embodiment, provided herein is a sterilized pharmaceutical composition comprising unfractionated bovine intestinal heparin and a pharmaceutically acceptable excipient, wherein the unfractionated bovine intestinal heparin has an anti-factor IIa activity of less than 100 U/mg as measured by USP methods. In certain embodiments, the sterilized pharmaceutical composition is an aqueous composition, such as in a buffered saline solution. In certain embodiments, the sterilized pharmaceutical composition is a dry powder formulation, such as a dry powder for inhalation. In certain embodiments, the sterilized pharmaceutical composition is formulated for administration with a nebulizer.

### DETAILED DESCRIPTION

All numerical designations, e.g., pH, temperature, time, concentration, and molecular weight, including ranges, are approximations which are varied (+) or (−) by increments of 0.1 or 10%. It is to be understood, although not always explicitly stated that all numerical designations are preceded by the term “about”. It also is to be understood, although not always explicitly stated, that the reagents described herein are merely exemplary and that equivalents of such are known in the art.

A “subject” of diagnosis or treatment is an animal such as a mammal, including a human.

A “pharmaceutical composition” is intended to include the combination of an active agent with a carrier, inert or active, making the composition suitable for diagnostic or therapeutic use in vitro, in vivo or ex vivo.

“An effective amount” refers to the amount of an agent sufficient to induce a desired biological and/or therapeutic result. That result can be alleviation of the signs, symptoms, or causes of a disease, or any other desired alteration of a biological system.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art.

The agents and compositions of the present invention can be used in the manufacture of medicaments and for the treatment of humans and other animals by administration in accordance with conventional procedures, such as an active ingredient in pharmaceutical compositions.

An agent of the present disclosure can be administered for therapy by any suitable route, such as by inhalation or parental (including subcutaneous, intramuscular, intravenous and intradermal) administration. It will also be appreciated that the preferred route will vary with the condition and age of the recipient, and the disease being treated.

### Treatment of Infections by Coronavirus

Recent studies have shown that heparin, a commonly used anticoagulant, may be useful in treating COVID-19, a disease caused by the infection of SARS-CoV-2, as well as other coronaviral infections. Heparin may bind to the spike protein of the virus and inhibit its entry into target cells. It is further contemplated that the heparin disclosed herein, and compositions comprising the same, may also be useful for treating various infections by coronaviruses, including Middle East Respiratory Syndrome (MERS)-CoV, SARS-CoV, SARS-CoV2, or a variant thereof. A variant includes coronaviruses identified as having an altered spike protein amino acid sequence from the original coronavirus. Exemplary variants include, but are not limited to, the B.1.1.7, B.1.351, P.1, B.1.427, and B.1.429 SARS-CoV-2 variants.

It has been suggested that heparin's anticoagulation activity is inherently coupled to its antiviral activities. For instance, Kim and colleagues (Kim et al., bioRxiv 2020.04.14.041459; doi: doi.org/10.1101/2020.04.14.041459) have demonstrated that N, 2-O, and 6-O sulfations are required for spike protein binding. Sulfation at these sites is also key to heparin's anticoagulation activity. The instant disclosure, however, shows that not all of the sulfation in heparin is required for its antiviral activity. As demonstrated in Example 1, IHP001, a bovine intestine heparin sample having about 20% reduction of 6-O desulfation as compared to the conventional unfractionated heparin (UFH), was as effective as the UFH in inhibiting infection of SARS-CoV into ACE2+ cells. Interestingly, IHP001's anticoagulation activity was less than 50% of the UFH. Such reduced anticoagulation activity, therefore, can allow a higher dose of the partially desulfated heparin to be used in patients.

Therefore, the instant inventor contemplates the use of a heparin molecule with reduced sulfation for the treatment of coronavirus infection. Compared to the standard heparin, i.e., unfractionated porcine intestine heparin (UPIH) as described in the United States Pharmacopeia (USP), certain heparins from non-standard sources (e.g., bovine) or chemically modified heparin variants can have reduced sulfation. Therefore, such heparins, referred to as “partially desulfated heparins,” can provide a more efficacious antiviral therapy than the standard heparin without increased anticoagulant activity which may not be desired in certain situations.

Heparin is a naturally occurring glycosaminoglycan. Glycosaminoglycans (GAGs) or mucopolysaccharides are long linear polysaccharides consisting of repeating disaccharide units. Except for keratan, the repeating unit consists of an amino sugar, along with a uronic sugar or galactose.

Native heparins have a molecular weight ranging from 3 to 30 kDa. The main disaccharide units that occur in heparin include GlcA-GlcNAc, GlcA-GlcNS, IdoA-GlcNS, IdoA(2S)-GlcNS, IdoA-GlcNS(6S), and IdoA(2S)-GlcNS(6S). GlcA denotes β-D-glucuronic acid; IdoA denotes α-L-iduronic acid; IdoA(2S) denotes 2-O-sulfo-α-L-iduronic acid; GlcNAc denotes 2-deoxy-2-acetamido-α-D-glucopyranosyl; GlcNS denotes 2-deoxy-2-sulfamido-α-D-glucopyranosyl; and GlcNS(6S) denotes 2-deoxy-2-sulfamido-α-D-glucopyranosyl-6-O-sulfate. The most common disaccharide unit in heparin is composed of a 2-O-sulfated iduronic acid and 6-O-sulfated, N-sulfated glucosamine, IdoA(2S)-GlcNS(6S).

Heparin binds to the enzyme inhibitor antithrombin III (AT), causing a conformational change that results in its activation through an increase in the flexibility of its reactive site loop. The activated AT then inactivates thrombin, factor Xa and other proteases. The rate of inactivation of these proteases by AT can increase by up to 1000-fold due to the binding of heparin. Heparin binds to AT via a specific pentasaccharide sulfation sequence contained within the heparin polymer: GlcNAc/NS(6S)-GlcA-GlcNS(3S,6S)-IdoA(2S)-GlcNS(6S).

The conformational change in AT on heparin-binding mediates its inhibition of factor Xa. For thrombin (factor IIa) inhibition, however, thrombin must also bind to the heparin polymer at a site proximal to the pentasaccharide. The highly negative charge density of heparin contributes to its very strong electrostatic interaction with thrombin. The formation of a ternary complex between AT, thrombin, and heparin results in the inactivation of thrombin. The anticoagulation activity of heparin can be measured in the lab by the partial thromboplastin time (aPTT), one of the measures of the time it takes the blood plasma to clot.

The sulfation level of the heparin may be associated with its anticoagulation activity. The sulfation level of a heparin molecule can be measured at the total number of S atoms in the molecule. More functionally direct perhaps, in some embodiments, the sulfation level is measured as the % number or mass of trisulfated disaccharide units. For instance, about 67% (w/w) to 69% (w/w) of disaccharide units in an unfractionated porcine intestine heparin (UPIH, a USP standard) are trisulfated. By contrast, about 51% (w/w) to 56% (w/w) of disaccharide units in the unfractionated bovine intestine porcine are trisulfated. Compared to the UPIH, therefore, bovine intestine heparin has an about 14.5 percentage points (about 21%) reduction at trisulfation level.

The term “trisulfation,” as used herein, refers to a disaccharide unit of a heparin molecule sulfated at three positions. A common example is ΔUA2S-GlcNS6S, where ΔUA denotes 4-deoxy-α-L-threo-hex-4-enopyranosyluronic acid. A specific example is IdoA(2S)-GlcNS(6S).

Alternatively, the sulfation level is measured as the % number or mass of disaccharide units having 6-O-sulfation. For instance, only about 5% (w/w) to 7% (w/w) of disaccharide units in the UPIH do not have 6-O-sulfation (having NS2S (ΔUA2S-GlcNS) instead). By contrast, about 15% (w/w) to 32% (w/w) of disaccharide units in the unfractionated bovine intestine are NS2S (or, do not have 6-O-sulfation). Compared to the UPIH, therefore, bovine intestine heparin has an about 17.5 percentage points (about 19%) reduction at 6-O-sulfation level.

Sulfation, or sulfated disaccharide units, can be measured with methods known in the art, such as HPLC-MS and HPLC-UV. A heparin with lower sulfation than the UPIH is also referred to as a “partially desulfated” heparin.

In accordance with one embodiment of the present disclosure, provided is a method for treating an infection by a coronavirus in a subject in need thereof. In some embodiments, the method entails administering to the subject an effective amount of a heparin having reduced sulfation as compared to unfractionated porcine intestine heparin (UPIH). In some embodiments, the method entails administering to the subject an effective amount of a bovine heparin having reduced sulfation as compared to unfractionated porcine intestine heparin (UPIH).

The term “heparin” also includes salts, such as heparin lithium, sodium, potassium, calcium, and the like. In some embodiments, the heparin is synthetic. In some embodiments, the heparin is a natural product and is not chemically modified. In some embodiments, the heparin is unfractionated. In some embodiments, the heparin is bovine intestine heparin. In some embodiments, the heparin is unfractionated bovine intestine heparin. In some embodiments, the heparin is non-chemically modified unfractionated bovine intestine heparin. Chemical modifications to heparin include, for example, sulfation, desulfation, or structural modifications which can occur as a result of a polymerization or depolymerization process, which process could result in a unnatural variation of the structure or composition of the native heparin. As such, in certain embodiments, the heparin is not obtained via a beta-elimination reaction (chemical or enzymatic), a deaminative cleavage reaction, and is not chemoenzymatically synthesized.

In certain embodiments, the unfractionated bovine intestinal heparin is not low molecular weight heparin, or does not comprise a substantial amount of low molecular weight heparin (e.g., heparin with a molecular weight of 4-6 kDa, or about 5 kDa, or less than 5 kDa, or less than 6 kDa). In certain embodiments, the unfractionated bovine intestinal heparin comprises less than 10%, or less than 8%, or less than 7%, or less than 6%, or less than 5%, or less than 4%, or less than 3%, or less than 2%, or less than 1%, of heparin with a molecular weight of less than 5 KDa, or less than 6 kDa. In certain embodiments, the sterilized pharmaceutical composition comprises unfractionated bovine intestinal heparin having an average molecular weight greater than 10 kDa to 20 kDa. In some embodiments, the heparin has an average molecular weight of greater than 15 kDa, or about 16 kDa, or about 17 kDa, or from 10 kDa to 18 kDa, or from 10 kDa to 19 kDa, or from 15 kDa to 20 kDa, or from 16 kDa to 18 kDa, or from 16 kDa to 17 kDa.

In some embodiments, as compared to the UPIH, the heparin has from 5% to 50% reduction of total sulfation. In some embodiments, the reduction is from 5% to 45%, 5% to 40%, 5% to 35%, 5% to 30%, 5% to 25%, 5% to 20%, 5% to 15%, 5% to 10%, 8% to 45%, 8% to 40%, 8% to 35%, 8% to 30%, 8% to 25%, 8% to 20%, 8% to 15%, 8% to 10%, 10% to 45%, 10% to 40%, 10% to 35%, 10% to 30%, 10% to 25%, 10% to 20%, 10% to 15%, 15% to 45%, 15% to 40%, 15% to 35%, 15% to 30%, 15% to 25%, 15% to 20%, 20% to 45%, 20% to 40%, 20% to 35%, 20% to 30%, 20% to 25%, 25% to 45%, 25% to 40%, 25% to 35%, or 25% to 30%.

In some embodiments, as compared to the UPIH, the heparin has from 5% to 50% reduction of trisulfation, such as ΔUA2S-GlcNS6S. In some embodiments, the reduction of trisulfation, such as ΔUA2S-GlcNS6S, is from 5% to 45%, 5% to 40%, 5% to 35%, 5% to 30%, 5% to 25%, 5% to 20%, 5% to 15%, 5% to 10%, 8% to 45%, 8% to 40%, 8% to 35%, 8% to 30%, 8% to 25%, 8% to 20%, 8% to 15%, 8% to 10%, 10% to 45%, 10% to 40%, 10% to 35%, 10% to 30%, 10% to 25%, 10% to 20%, 10% to 15%, 15% to 45%, 15% to 40%, 15% to 35%, 15% to 30%, 15% to 25%, 15% to 20%, 20% to 45%, 20% to 40%, 20% to 35%, 20% to 30%, 20% to 25%, 25% to 45%, 25% to 40%, 25% to 35%, or 25% to 30%.

In some embodiments, the total amount of trisulfated disaccharide units, such as ΔUA2S-GlcNS6S, is from 20% w/w to 65% w/w of all the disaccharide units in the heparin. In some embodiments, the total amount of trisulfated disaccharide units, such as ΔUA2S-GlcNS6S, is 20% w/w to 65% w/w, 20% w/w to 60% w/w, 20% w/w to 55% w/w, 20% w/w to 50% w/w, 20% w/w to 45% w/w, 20% w/w to 40% w/w, 20% w/w to 35% w/w, 20% w/w to 30% w/w, 20% w/w to 25% w/w, 25% w/w to 65% w/w, 25% w/w to 60% w/w, 25% w/w to 55% w/w, 25% w/w to 50% w/w, 25% w/w to 45% w/w, 25% w/w to 40% w/w, 25% w/w to 35% w/w, 25% w/w to 30% w/w, 30% w/w to 65% w/w, 30% w/w to 60% w/w, 30% w/w to 55% w/w, 30% w/w to 50% w/w, 30% w/w to 45% w/w, 30% w/w to 40% w/w, 30% w/w to 35% w/w, 35% w/w to 65% w/w, 35% w/w to 60% w/w, 35% w/w to 55% w/w, 35% w/w to 50% w/w, 35% w/w to 45% w/w, 35% w/w to 40% w/w, 40% w/w to 65% w/w, 40% w/w to 60% w/w, 40% w/w to 55% w/w, 40% w/w to 50% w/w, 40% w/w to 45% w/w, 45% w/w to 65% w/w, 45% w/w to 60% w/w, 45% w/w to 55% w/w, 45% w/w to 50% w/w, 50% w/w to 65% w/w, 50% w/w to 60% w/w, 50% w/w to 55% w/w, 55% w/w to 65% w/w, or 55% w/w to 60% w/w.

In some embodiments, the total number of trisulfated disaccharide units, such as ΔUA2S-GlcNS6S, is from 20% to 65% (by count) of all the disaccharide units in the heparin. In some embodiments, the total number of trisulfated disaccharide units, such as ΔUA2S-GlcNS6S, is 20% to 65%, 20% to 60%, 20% to 55%, 20% to 50%, 20% to 45%, 20% to 40%, 20% to 35%, 20% to 30%, 20% to 25%, 25% to 65%, 25% to 60%, 25% to 55%, 25% to 50%, 25% to 45%, 25% to 40%, 25% to 35%, 25% to 30%, 30% to 65%, 30% to 60%, 30% to 55%, 30% to 50%, 30% to 45%, 30% to 40%, 30% to 35%, 35% to 65%, 35% to 60%, 35% to 55%, 35% to 50%, 35% to 45%, 35% to 40%, 40% to 65%, 40% to 60%, 40% to 55%, 40% to 50%, 40% to 45%, 45% to 65%, 45% to 60%, 45% to 55%, 45% to 50%, 50% to 65%, 50% to 60%, 50% to 55%, 55% to 65%, or 55% to 60%.

In some embodiments, as compared to the UPIH, the heparin has from 5% to 50% reduction of 6-O-sulfated disaccharide units. In some embodiments, the reduction of 6-O-sulfated disaccharide units is from 5% to 45%, 5% to 40%, 5% to 35%, 5% to 30%, 5% to 25%, 5% to 20%, 5% to 15%, 5% to 10%, 8% to 45%, 8% to 40%, 8% to 35%, 8% to 30%, 8% to 25%, 8% to 20%, 8% to 15%, 8% to 10%, 10% to 45%, 10% to 40%, 10% to 35%, 10% to 30%, 10% to 25%, 10% to 20%, 10% to 15%, 15% to 45%, 15% to 40%, 15% to 35%, 15% to 30%, 15% to 25%, 15% to 20%, 20% to 45%, 20% to 40%, 20% to 35%, 20% to 30%, 20% to 25%, 25% to 45%, 25% to 40%, 25% to 35%, or 25% to 30%.

In some embodiments, the total amount of 6-O-sulfated disaccharide units is from 50% w/w to 80% w/w of all the disaccharide units in the heparin. In some embodiments, the total amount of 6-O-sulfated disaccharide units is from 35% w/w to 80% w/w, 40% w/w to 80% w/w, 45% w/w to 80% w/w, or 50% w/w to 80% w/w, of all the disaccharide units in the heparin. In some embodiments, the total amount of trisulfated disaccharide units is 50% w/w to 80% w/w, 50% w/w to 75% w/w, 50% w/w to 70% w/w, 50% w/w to 65% w/w, 50% w/w to 60% w/w, 55% w/w to 80% w/w, 55% w/w to 75% w/w, 55% w/w to 70% w/w, 55% w/w to 65% w/w, 55% w/w to 60% w/w, 60% w/w to 80% w/w, 60% w/w to 75% w/w, 60% w/w to 70% w/w, 60% w/w to 65% w/w, 65% w/w to 80% w/w, 65% w/w to 75% w/w, 65% w/w to 70% w/w, 70% w/w to 80% w/w, 70% w/w to 75% w/w, or 75% w/w to 80% w/w.

In some embodiments, the total number of 6-O-sulfated disaccharide units is from 50% to 80% (by count) of all the disaccharide units in the heparin. In some embodiments, the total number of 6-O-sulfated disaccharide units is from 35% w/w to 80% w/w, 40% w/w to 80% w/w, 45% w/w to 80% w/w, or 50% w/w to 80% w/w (by count) of all the disaccharide units in the heparin. In some embodiments, the total number of trisulfated disaccharide units is 50% to 80%, 50% to 75%, 50% to 70%, 50% to 65%, 50% to 60%, 55% to 80%, 55% to 75%, 55% to 70%, 55% to 65%, 55% to 60%, 60% to 80%, 60% to 75%, 60% to 70%, 60% to 65%, 65% to 80%, 65% to 75%, 65% to 70%, 70% to 80%, 70% to 75%, or 75% to 80%.

The anticoagulant activity of the heparin can also be measured with respect to its activity to inhibit factor Xa (fXa) or factor IIa (thrombin). An example can be found in, e.g., Stuart, M, Johnson, L, Hanigan, S, Pipe, SW, Li, S-H. Anti-factor IIa (FIIa) heparin assay for patients on direct factor Xa (FXa) inhibitors. J Thromb Haemost. 2020; 00:1-8 (doi.org/10.1111/jth.14806).

In one embodiment, therefore, the present disclosure provides a method for treating an infection by a coronavirus in a subject in need thereof. In some embodiments, the method entails administering to the subject an effective amount of a heparin having from 10% to 70% reduction of anti-factor IIa (Ma) or anti-factor Xa (fXa) activity as compared to unfractionated porcine intestine heparin (UPIH).

In some embodiments, as compared to the UPIH, the heparin has 10% to 70%, or 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 70%, 20% to 65%, 20% to 60%, 20% to 55%, 20% to 50%, 20% to 45%, 20% to 40%, 20% to 35%, 20% to 30%, 20% to 25%, 25% to 70%, 25% to 65%, 25% to 60%, 25% to 55%, 25% to 50%, 25% to 45%, 25% to 40%, 25% to 35%, 25% to 30%, 30% to 70%, 30% to 65%, 30% to 60%, 30% to 55%, 30% to 50%, 30% to 45%, 30% to 40%, 30% to 35%, 35% to 70%, 35% to 65%, 35% to 60%, 35% to 55%, 35% to 50%, 35% to 45%, 35% to 40%, 40% to 70%, 40% to 65%, 40% to 60%, 40% to 55%, 40% to 50%, 40% to 45%, 45% to 70%, 45% to 65%, 45% to 60%, 45% to 55%, 45% to 50%, 50% to 70%, 50% to 65%, 50% to 60%, 50% to 55%, 55% to 70%, 55% to 65%, 55% to 60%, 60% to 70%, 60% to 65% or 65% to 70% reduction of anti-fila activity.

In some embodiments, the heparin has an anti-fila activity of 70 U/mg to 140 U/mg, 70 U/mg to 130 U/mg, 70 U/mg to 120 U/mg, 70 U/mg to 110 U/mg, 70 U/mg to 100 U/mg, 70 U/mg to 90 U/mg, 75 U/mg to 140 U/mg, 75 U/mg to 130 U/mg, 75 U/mg to 120 U/mg, 75 U/mg to 110 U/mg, 75 U/mg to 105 U/mg, 75 U/mg to 100 U/mg, 75 U/mg to 90 U/mg, 80 U/mg to 140 U/mg, 80 U/mg to 130 U/mg, 80 U/mg to 120 U/mg, 80 U/mg to 110 U/mg, 80 U/mg to 100 U/mg, 80 U/mg to 90 U/mg, 85 U/mg to 140 U/mg, 85 U/mg to 130 U/mg, 85 U/mg to 120 U/mg, 85 U/mg to 110 U/mg, 85 U/mg to 105 U/mg, 85 U/mg to 100 U/mg, 85 U/mg to 90 U/mg, 90 U/mg to 140 U/mg, 90 U/mg to 130 U/mg, 90 U/mg to 120 U/mg, 90 U/mg to 110 U/mg, 90 U/mg to 100 U/mg, 100 U/mg to 140 U/mg, 100 U/mg to 130 U/mg, 100 U/mg to 120 U/mg, 100 U/mg to 110 U/mg, 110 U/mg to 140 U/mg, 110 U/mg to 130 U/mg, 110 U/mg to 120 U/mg, 120 U/mg to 140 U/mg, 120 U/mg to 130 U/mg, or 130 U/mg to 140 U/mg. In some embodiments, the heparin is unfractionated bovine intestinal heparin having an anti-factor IIa activity of from 70 to less than 100 U/mg, 75 to less than 100 U/mg, 80 to less than 100 U/mg, 82 to less than 100 U/mg, 84 to less than 100 U/mg, 86 to less than 100 U/mg, 88 to less than 100 U/mg, 90 to less than 100 U/mg, 91 to less than 100 U/mg, 92 to less than 100 U/mg, 93 to less than 100 U/mg, 94 to less than 100 U/mg, 95 to less than 100 U/mg, 96 to less than 100 U/mg, 97 to less than 100 U/mg, about 90 U/mg, about 91 U/mg, about 92 U/mg, about 93 U/mg, about 94 U/mg, about 95 U/mg, about 96 U/mg, about 97 U/mg, about 98 U/mg, less than about 95 U/mg, less than about 96 U/mg, less than about 97 U/mg, less than about 98 U/mg, less than about 99 U/mg, or less than about 100 U/mg, as measured by USP methods.

In some embodiments, the heparin has an anti-factor Xa to anti-factor IIa ratio of from 0.9-1.1, or about 1 or 1.

In some embodiments, as compared to the UPIH, the heparin has 10% to 70%, or 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 70%, 20% to 65%, 20% to 60%, 20% to 55%, 20% to 50%, 20% to 45%, 20% to 40%, 20% to 35%, 20% to 30%, 20% to 25%, 25% to 70%, 25% to 65%, 25% to 60%, 25% to 55%, 25% to 50%, 25% to 45%, 25% to 40%, 25% to 35%, 25% to 30%, 30% to 70%, 30% to 65%, 30% to 60%, 30% to 55%, 30% to 50%, 30% to 45%, 30% to 40%, 30% to 35%, 35% to 70%, 35% to 65%, 35% to 60%, 35% to 55%, 35% to 50%, 35% to 45%, 35% to 40%, 40% to 70%, 40% to 65%, 40% to 60%, 40% to 55%, 40% to 50%, 40% to 45%, 45% to 70%, 45% to 65%, 45% to 60%, 45% to 55%, 45% to 50%, 50% to 70%, 50% to 65%, 50% to 60%, 50% to 55%, 55% to 70%, 55% to 65%, 55% to 60%, 60% to 70%, 60% to 65% or 65% to 70% reduction of anti-fXa activity.

The presently disclosed treatments can be useful for treating various infections by coronaviruses. In one embodiment, the coronavirus is Middle East Respiratory Syndrome (MERS)-CoV. In one embodiment, the coronavirus is SARS-CoV.

In one embodiment, the coronavirus is SARS-CoV2. In some embodiments, the subject has one or more symptoms of COVID-19, such as fever or chills, cough, shortness of breath or difficulty breathing, fatigue, muscle or body aches, headache, new loss of taste or smell, sore throat, congestion or runny nose, nausea or vomiting, or diarrhea.

In one embodiment, the subject is hospitalized. In one embodiment, the subject is not hospitalized. In one embodiment, the subject is treated with mechanical ventilation. In one embodiment, the subject suffers from a cytokine release syndrome.

Combinations and Pharmaceutical Compositions

In one embodiment, the treatment methods can further include administration of an effective amount of another agent. In some embodiments, the other agent is an anti-spike protein antibody or fragment. In some embodiments, the second agent is co-administered with the antibody or fragment thereof simultaneously or sequentially.

In some embodiments, the second agent is effective in reducing or inhibiting cytokine release storm. In some embodiments, the second agent is a corticosteroid. Non-limiting examples include methylprednisolone (in particular in patients with a rheumatic disease), dexamethasone (in particular in patients with FHLH).

In some embodiments, the second agent is a cytoablative therapy. Non-limiting examples include cyclophosphamide (in particular in patients with JIA and MAS), etoposide (in particular in patients with FHLH), rituximab (in particular in Epstein-Barr virus (EBV)-associated HLH), antithymocyte globulin (in particular for patients at bone marrow transplant phase of FHLH therapy), alemtuzumab (in particular in patients with FHLH or SLE-associated MAS).

In some embodiments, the second agent is a T-cell modulator. Non-limiting examples include calcineurin (e.g., cyclosporine) which prevents production of IL-2, and abatacept, which inhibits CD28 signaling of T cells. In some embodiments, the second agent is an anti-GM-CSF inhibitor or antibody.

In some embodiments, the second agent is a cytokine inhibitor, such inhibitors targeting INFγ, IL-1β, IL-18, IL-33, IL-6, and/or TNF.

In some embodiments, the second agent targets the underlying disease or condition, such as SARS-CoV-2 infection. Non-limiting examples include lopinavir, ritonavir, oseltamivir (Tamiflu), favipiravir, fingolimod, methylprednisolone, bevacizumab, chloroquine phosphate, chloroquine, hydroxychloroquine sulfate and remdesivir.

In another aspect, the present disclosure provides a pharmaceutical composition comprising a heparin of the present disclosure formulated together with a pharmaceutically acceptable earlier. It may optionally contain one or more additional pharmaceutically active ingredients, such as a heparin or a drug. The pharmaceutical compositions of the disclosure also can be administered in a combination therapy with, for example, an anti-viral agent, or a vaccine.

In certain embodiments, the additional therapeutic agent is convalescent plasma, a vaccine, a corticosteroid (e.g., dexamethasone, hydrocortisone, methylprednisolone, etc.), baricitinib, a monoclonal antibody (MAB) (e.g., bamlanivimab (LY-CoV555), casirivimab imdevimab (REGN-COV2), etc.), and/or remdesivir.

In certain embodiments, the additional therapeutic agent is hydroxychloroquine, chloroquine, azithromycin, an IL-6 inhibitor (e.g., tocilizumab, sarilumab, etc.), kinase inhibitor (e.g., Acalabrutinib (Calquence), Baricitinib (Olumiant), Ruxolitinib (Jakafi), Tofacitinib (Xeljanz), etc.), interferon-alfa (IFN-α), interferon-beta (IFN-β), Kaletra (lopinavir/ritonavir), ivermectin, Tamiflu (oseltamivir), favipiravir, umifenovir, galidesivir, colchicine, convalescent plasma, a corticosteroid (e.g., dexamethasone, hydrocortisone, methylprednisolone, etc.), baricitinib, a monoclonal antibody (MAB) (e.g., bamlanivimab (LY-CoV555) casirivimab, imdevimab (REGN-COV2), etc.), Alpha-1 antitrypsin, or remdesivir.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference. In certain embodiments, a pharmaceutical composition is suitable for intravenous, intramuscular, subcutaneous, parenteral, spinal or epidermal administration (e.g., by injection or infusion). Depending on the route of administration, the active compound can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Alternatively, a heparin of the disclosure can be administered via a non-parenteral route, such as a topical, epidermal or mucosal route of administration, e.g., intranasally, orally, vaginally, rectally, sublingually or topically. An exemplary non-parenteral route includes administration via inhalation.

Pharmaceutical compositions can be in the form of sterile aqueous solutions or dispersions. They can also be formulated in a microemulsion, liposome, or other ordered structure suitable to high drug concentration.

In certain embodiments, provided herein is a sterilized pharmaceutical composition comprising unfractionated bovine intestinal heparin and a pharmaceutically acceptable excipient, wherein the unfractionated bovine intestinal heparin has an anti-factor IIa activity of less than 100 U/mg as measured by USP methods. In certain embodiments, the sterilized pharmaceutical composition has a pH of about 6-8, or about 7.2. In certain embodiments, the sterilized pharmaceutical composition is an aqueous composition, such as in a buffered saline solution. In certain embodiments, the sterilized pharmaceutical composition is a dry powder formulation, such as a dry powder for inhalation.

In certain embodiments, the unfractionated bovine intestinal heparin is not chemically modified. Chemical modifications are described in the art, as well as hereinabove. In certain embodiments, the unfractionated bovine intestinal heparin is not low molecular weight heparin, or does not comprise a substantial amount of low molecular weight heparin. In certain embodiments, the unfractionated bovine intestinal heparin comprises less than 10%, or less than 8%, or less than 7%, or less than 6%, or less than 5%, or less than 4%, or less than 3%, or less than 2%, or less than 1%, heparin less than 5 KDa. In certain embodiments, the sterilized pharmaceutical composition comprises unfractionated bovine intestinal heparin having an average molecular weight greater than 10 KDa, greater than 15 KDa, of from 15 KDa to 20 KDa, or from 16 KDa to 17 KDa, or about 16 KDa.

In certain embodiments, the sterilized pharmaceutical composition comprises unfractionated bovine intestinal heparin has about 20% reduction of 6-O desulfation as compared to conventional unfractionated heparin (UFH), such as unfractionated porcine intestinal heparin. In some embodiments, as compared to the UPIH, the heparin has from 5% to 50% reduction of 6-O-sulfated disaccharide units. In some embodiments, the reduction of 6-O-sulfated disaccharide units is from 5% to 45%, 5% to 40%, 5% to 35%, 5% to 30%, 5% to 25%, 5% to 20%, 5% to 15%, 5% to 10%, 8% to 45%, 8% to 40%, 8% to 35%, 8% to 30%, 8% to 25%, 8% to 20%, 8% to 15%, 8% to 10%, 10% to 45%, 10% to 40%, 10% to 35%, 10% to 30%, 10% to 25%, 10% to 20%, 10% to 15%, 15% to 45%, 15% to 40%, 15% to 35%, 15% to 30%, 15% to 25%, 15% to 20%, 20% to 45%, 20% to 40%, 20% to 35%, 20% to 30%, 20% to 25%, 25% to 45%, 25% to 40%, 25% to 35%, or 25% to 30%.

Degree of sulfation can be determined by conventional means, such as NMR and/or disaccharide analysis.

In certain embodiments, the sterilized pharmaceutical composition comprises unfractionated bovine intestinal heparin characterized by having less sulfation at one or more positions as compared to conventional unfractionated heparin (UFH), such as unfractionated porcine intestinal heparin, and/or conventional unfractionated bovine heparin.

In certain embodiments, the unfractionated bovine intestinal heparin characterized by having a lower amount of N,6-disulfated α-D-glucosamine (α-GlcN,6-diS). In certain embodiments, the unfractionated bovine intestinal heparin characterized by having a lower amount of N,6-disulfated α-D-glucosamine (α-GlcN,6-diS) attached to 2-sulfated α-iduronic acid (α-IdA2S) as determined by NMR. In certain embodiments, the unfractionated bovine intestinal heparin characterized by having less than 47% N,6-disulfated α-D-glucosamine (α-GlcN,6-diS) attached to 2-sulfated α-iduronic acid (α-IdA2S) as determined by NMR, or less than 46% N,6-disulfated α-D-glucosamine (α-GlcN,6-diS) attached to 2-sulfated α-iduronic acid (α-IdA2S) as determined by NMR, about 45% N,6-disulfated α-D-glucosamine (α-GlcN,6-diS) attached to 2-sulfated α-iduronic acid (α-IdA2S) as determined by NMR, about 44% N,6-disulfated α-D-glucosamine (α-GlcN,6-diS) attached to 2-sulfated α-iduronic acid (α-IdA2S) as determined by NMR.

In certain embodiments, the unfractionated bovine intestinal heparin characterized by having a lower amount of N,3,6-trisulfated α-D-glucosamine (α-GlcN,3,6-triS). In certain embodiments, the unfractionated bovine intestinal heparin characterized by having less than 4% N,3,6-trisulfated α-D-glucosamine (α-GlcN,3,6-triS) as determined by NMR, or less than 3% N,3,6-trisulfated α-D-glucosamine (α-GlcN,3,6-triS) as determined by NMR, or about 2% N,3,6-trisulfated α-D-glucosamine (α-GlcN,3,6-triS) as determined by NMR, or from 1-3% N,3,6-trisulfated α-D-glucosamine (α-GlcN,3,6-triS) as determined by NMR, or from 2-3% N,3,6-trisulfated α-D-glucosamine (α-GlcN,3,6-triS) as determined by NMR.

In certain embodiments, the sterilized pharmaceutical composition comprises unfractionated bovine intestinal heparin characterized by having one or more of less than 47% N,6-disulfated α-D-glucosamine (α-GlcN,6-diS) attached to 2-sulfated α-iduronic acid (α-IdA2S) as determined by NMR; or less than 2% N,3,6-trisulfated α-D-glucosamine (α-GlcN,3,6-triS) as determined by NMR. In certain embodiments, the sterilized pharmaceutical composition comprises unfractionated bovine intestinal heparin characterized by having one or more of less than 47% N,6-disulfated α-D-glucosamine (α-GlcN,6-diS) attached to 2-sulfated α-iduronic acid (α-IdA2S) as determined by NMR; or less than 3% N,3,6-trisulfated α-D-glucosamine (α-GlcN,3,6-triS) as determined by NMR. In certain embodiments, the sterilized pharmaceutical composition comprises unfractionated bovine intestinal heparin characterized by having one or more of less than 47% N,6-disulfated α-D-glucosamine (α-GlcN,6-diS) attached to 2-sulfated α-iduronic acid (α-IdA2S) as determined by NMR; or less than 4% N,3,6-trisulfated α-D-glucosamine (α-GlcN,3,6-triS) as determined by NMR.

In certain embodiments, the sterilized pharmaceutical composition comprises unfractionated bovine intestinal heparin having an anti-factor IIa activity of from 70 to less than 100 U/mg, 75 to less than 100 U/mg, 80 to less than 100 U/mg, 82 to less than 100 U/mg, 84 to less than 100 U/mg, 86 to less than 100 U/mg, 88 to less than 100 U/mg, 90 to less than 100 U/mg, 91 to less than 100 U/mg, 92 to less than 100 U/mg, 93 to less than 100 U/mg, 94 to less than 100 U/mg, 95 to less than 100 U/mg, 96 to less than 100 U/mg, 97 to less than 100 U/mg, about 90 U/mg, about 91 U/mg, about 92 U/mg, about 93 U/mg, about 94 U/mg, about 95 U/mg, about 96 U/mg, about 97 U/mg, about 98 U/mg, less than about 95 U/mg, less than about 96 U/mg, less than about 97 U/mg, less than about 98 U/mg, less than about 99 U/mg, or less than about 100 U/mg, as measured by USP methods.

In certain embodiments, the sterilized pharmaceutical composition, or the unfractionated bovine intestinal heparin, has an anti-factor Xa to anti-factor IIa ratio of from 0.9-1.1, or about 1 or 1.

In certain embodiments, the sterilized pharmaceutical composition is heat sterilized. In certain embodiments, the sterilized pharmaceutical composition is sterilized via filtration.

The pharmaceutical compositions described herein can comprise a higher dose of heparin due to the low anticoagulant or low anti-factor IIa activity of the composition. Accordingly, in certain embodiments, a sterilized pharmaceutical composition as described herein is prepared for administration at a final concentration of heparin greater than 50 mg/mL. However, the sterilized pharmaceutical compositions as described herein can be prepared at various concentration as needed, such as, but not limited to, from 10 mg/mL to 100 mg/mL, from 20 mg/mL to 100 mg/mL, from 30 mg/mL to 100 mg/mL, from 40 mg/mL to 100 mg/mL, from 50 mg/mL to 100 mg/mL, from greater than 50 mg/mL to 100 mg/mL, from 60 mg/mL to 100 mg/mL, from 70 mg/mL to 100 mg/mL, from 80 mg/mL to 100 mg/mL, from 90 mg/mL to 100 mg/mL, from 10 mg/mL to 100 mg/mL, from 20 mg/mL to 90 mg/mL, from 30 mg/mL to 80 mg/mL, from 40 mg/mL to 80 mg/mL, from greater than 50 mg/mL to 80 mg/mL, from 60 mg/mL to 80 mg/mL, from greater than 50 mg/mL to 70 mg/mL.

In certain embodiments, the sterilized pharmaceutical composition described herein is formulated for inhalation, continuous infusion, intravenous infusion, or subcutaneous administration. In certain embodiments, the pharmaceutical composition is formulated for administration via inhalation, orally or intranasally. As such, the pharmaceutical composition can be administered with a dry powder inhaler, metered dose inhaler, pressurized metered dose inhaler, nebulizer, or soft mist inhaler. In certain embodiments, the sterilized pharmaceutical composition is an aqueous composition. In certain embodiments, the sterilized pharmaceutical composition comprises a pharmaceutically acceptable excipient selected from a buffer, preservative, co-solvent, suspending agent, surfactant, tonicity adjusting agent, humectants, or combination thereof.

In certain embodiments, the pharmaceutical composition is administered with a nebulizer such as a high efficiency nebulizer. Nebulizer formulations are typically aqueous-based sterile formulations that contain therapeutically active ingredients and can also contain one or more additional excipients. Various excipients commonly used in nebulizer formulations include, buffer, preservative, co-solvent, suspending agent, surfactant, tonicity adjusting agent, humectants etc. Exemplary excipients for use in the pharmaceutical composition described herein include, but are not limited to, an isotonicity adjusting agent (e.g., sodium chloride, dextrose, etc.), a pH adjusting agent (e.g., sodium hydroxide, hydrochloric acid, sulfuric acid, etc.), a purging agent to reduce oxidation (e.g., nitrogen, etc.), an antimicrobial preservative (e.g., benzalkonium chloride, ethanol, propylene glycol, benzoyl alcohol, chlorobutanol, methyl paraben, etc.), a buffer component (e.g., sodium citrate, sodium phosphate, citric acid, etc.), a surfactant (e.g., polysorbate 80, polysorbate 20, etc.), a cation chelating agent (e.g., disodium EDTA, etc.), a suspending agent or viscosity increasing agent (e.g., CMC, Na CMC, etc.), a co-solvent (alcohol, PEG 400, propylene glycol, etc.) or a humectant (e.g., glycerin).

The droplet size distribution of a nebulizer is a parameter which can influence the in vivo deposition of the heparin in the lung. The droplet size can be influenced by the formulation, the nebulizer device, or both. In certain embodiments, the delivered atomized particles are comprised of particles substantially having a mean diameter of, for example, from 1 μm to 30 μm, 5 μm to 30 μm, 10 μm to 30 μm, 15 μm to 30 μm, 10 μm to 20 μm, 10 μm to 15 μm, 1 μm to 20 μm, 1 μm to 10 μm, less than 30 μm, less than 25 μm, less than 20 μm, less than 15 μm, less than 10 μm, less than 9 μm, less than 8 μm, less than 7 μm, less than 6 μm, less than 5 μm, less than 4 μm, less than 3 μm, less than 2 μm, or less than 1 μm.

In certain embodiments, the pharmaceutical composition is formulated for dry powder inhalation. A dry powder inhalation (DPI) formulation is a dosage form containing micronized drug particles which comprise a heparin of the disclosure that are small enough to be deposited in the lungs. Methods and compositions for making suitable DPI formulations are known in the art. For example, a spray-drying procedure is frequently applied as a method for DPI formulation. Using ethanol as co-solvent can help to produce micronized systems. Various excipients can also be employed to modify the surface and stabilizing particle size, such as polyvinyl alcohol, L-leucine, cyclodextrin, and the like. In certain embodiments, the dry powder inhalation (DPI) formulation comprises lactose. In certain embodiments, the dry powder inhalation (DPI) formulation does not comprise lactose. Exemplary dry powder inhalation (DPI) formulation for use in the methods disclosed herein are shown below in Table 1.

TABLE 1

| | Heparin | LEU | PVA | CD |
|---|---|---|---|---|
| No. | (g) | (g) | (g) | (g) |
| 1 | 1 | — | — | — |
| 2 | 1 | — | 0.2 | — |
| 3 | 1 | — | — | 0.9 |
| 4 | 1 | 0.4 | — | — |
| 5 | 1 | 0.4 | 0.2 | 0.9 |

PVA: polyvinyl alcohol; LEU: L-leucine; CD: cyclodextrin.

In certain embodiments, the dry powder inhalation (DPI) formulation comprises micronized drug particles having a mean diameter of, for example, from 1 μm to 30 μm, 5 μm to 30 μm, 10 μm to 30 μm, 15 μm to 30 μm, 10 μm to 20 μm, 10 μm to 15 μm, 1 μm to 20 μm, 1 μm to 10 μm, 1 μm to 9 μm, 1 μm to 8 μm, 1 μm to 7 μm, 1 μm to 6 μm, 1 μm to 5 μm, 2 μm to 10 μm, 2 μm to 9 μm, 2 μm to 8 μm, 2 μm to 7 μm, 2 μm to 6 μm, 2 μm to 5 μm, less than 30 μm, less than 25 μm, less than 20 μm, less than 15 μm, less than 10 μm, less than 9 μm, less than 8 μm, less than 7 μm, less than 6 μm, less than 5 μm, less than 4 μm, less than 3 μm, less than 2 μm, or less than 1 μm.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the heparin can be administered as a sustained release formulation, in which case less frequent administration is required.

For administration of the heparin, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every 3 to 6 months. Preferred dosage regimens for a heparin of the disclosure include 1 mg/kg body weight or 3 mg/kg body weight via intravenous administration, with the heparin being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma heparin concentration of about 1-1000 μg/mL and in some methods about 25-300 μg/mL.

In some embodiments, a suitable dose of a heparin of the disclosure for a human patient is from 5 mg to 1200 mg, from 10 mg to 1000 mg, from 20 mg to 800 mg, from 50 mg to 800 mg, from 100 mg to 800 mg, from 150 mg to 800 mg, from 200 mg to 600 mg, or from 300 mg to 500 mg daily. In some embodiments, a suitable dose of a heparin of the disclosure for a human patient is about 5 mg, 10 mg, 20 mg, 40 mg, 50 mg, 100 mg, 200 mg, 300 mg, 400 mg, 500 mg, 600 mg, 700 mg, 800 mg, 900 mg, 1000 mg, 1100 mg, or 1200 mg daily. In some embodiments, a suitable dose of a heparin of the disclosure for a human patient is about 400 mg daily.

In some embodiments, the patient has an infection with severe symptoms. A suitable dose of a heparin of the disclosure for this patient is, in some embodiments, 20 mg to 1200 mg, from 40 mg to 1000 mg, from 50 mg to 800 mg, from 75 mg to 800 mg, from 100 mg to 800 mg, from 150 mg to 800 mg, from 200 mg to 600 mg, or from 300 mg to 500 mg daily. In some embodiments, a suitable dose of a heparin of the disclosure is about 20 mg, 40 mg, 50 mg, 100 mg, 200 mg, 300 mg, 400 mg, 500 mg, 600 mg, 700 mg, 800 mg, 900 mg, 1000 mg, 1100 mg, or 1200 mg daily.

In some embodiments, the patient has an infection with mile symptoms or is at risk of developing an infection. A suitable dose of a heparin of the disclosure for this patient is, in some embodiments, 5 mg to 600 mg, from 10 mg to 500 mg, from 20 mg to 400 mg, from 30 mg to 400 mg, from 40 mg to 400 mg, from 50 mg to 400 mg, from 50 mg to 300 mg, from 50 mg to 250 mg daily, from 70 mg to 200 mg, from 80 mg to 200, from 100 mg to 200 mg, from 120 mg to 180 mg, or from 140 mg to 160 mg. In some embodiments, a suitable dose of a heparin of the disclosure is about 20 mg, 40 mg, 50 mg, 100 mg, 150 mg, 200 mg, 300 mg, or 400 mg daily.

In some embodiments, the administration is once, twice or three times a day.

### EXAMPLES

### Example 1. Heparin Compositions

Heparin purified from bovine intestinal tissues following methods such as those published in [der Meer, Van, Edwin Kellenbach, and Leendert J. Van den Bos. “From farm to pharma: an overview of industrial heparin manufacturing methods.” Molecules 22.6 (2017): 1025] can be used in the following methods. The following process is intended to be performed to obtain purified bovine intestinal heparin without substantially affecting the material by degradation (e.g., depolymerization and/or desulfation) or other chemical modifications caused by the applied process conditions.

Briefly, intestinal tissues are digested enzymatically (a wide range of proteolytic enzymes can be used including trypsin, chymotrypsin, papain or Alcalase® or Maxatase®). Heparin is then captured on an anionic resin to concentrate and separate it from non-glycosaminoglycan components. The heparin is then eluted from the anionic resin by salt solution, such as sodium chloride. The anticoagulant activity of heparin is related to how strongly heparin binds to the anion exchange resin. Methods have been employed to increase the anticoagulant activity by washing the lower activity fractions with a low ionic solution (<3.5% NaCl). Alternatively, to produce a lower anticoagulant heparin, an ionic wash step may not be performed and the salt concentration to elute heparin can be reduced to keep the highly sulfated fractions of heparin bound to the resin and thus not included in the final heparin product.

The eluted heparin may also contain non-heparin glycosaminoglycans and nucleic acids and can be purified by precipitation with organic solvents such as methanol, ethanol, propanol, or acetone. The precipitated heparin is then subjected to one or more purification processes, such as, but not limited to, bleaching, oxidation, basic media, to remove potential endotoxins, viruses, and prions. For example, viruses can be inactivated under basic conditions, such as pH>11 in sodium hydroxide. Oxidation can be performed using a suitable oxidant, such as potassium permanganate (KMnO₄), hydrogen peroxide (H₂O₂), peracetic acid (CH₃CO₃H), sodium hypochlorite (NaClO), or ozone (O₃). The purification process can be performed such that the heparin is not substantially altered (e.g., non-chemically modified). The resulting heparin is then purified by sterile filtration, and the purified heparin is again precipitated with alcohol and then dried.

To obtain the desired low anticoagulant heparin, batches of heparin can be measured for anti-factor IIa activity and then blended such that the final batch of heparin contains anti-factor IIa activity of <100 U/mg. Anti-factor IIa activity is measured by methods described in the heparin U.S. Pharmacopeia (USP methods). If batches of heparin are <100 U/mg, they can be used without blending with other batches. If a batch of heparin is >100 U/mg, it can be blended with other batches of heparin with anti-factor IIa levels <100 U/mg, provided the final blended batch is <100 U/mg.

For example, a 1 kg batch of heparin at 105 U/mg could be blended with 1 kg of heparin at 90 U/mg to yield a final batch of heparin with anti-factor IIa activity of 97.5 U/mg. In this way the desired reduced anticoagulant activity heparin product is produced. For clarity, these heparin batches can be sourced from either a single heparin API manufacturer, or from multiple heparin API manufacturers, and blended appropriately to achieve the desired anti-factor Ha activity of <100 U/mg for the final blended batch.

Formulation and Sterile Filtration

The low anticoagulant heparin prepared as described and provided as a dry powder is subsequently processed into a sterile solution formulation for administration to a patient. The heparin dry powder is dissolved into water for injection to a final concentration as intended based on either a mass basis or based on the anti-factor Ha activity. In one example, heparin is dissolved into water for injection at a concentration of 5,000 U/mL. Heparin dry powder can be added to water to dissolve to the desired concentration. For concentrated formulations, dissolution can be aided by heating, such as to about 40 ° C. with rigorous mixing. Once the heparin is dissolved, the solution can be brought to the desired tonicity, e.g., with sodium chloride. For example, sodium chloride can be added to reach a final osmolarity of 300 mOsm/L. Once the solution is brought to the desired osmolarity, the pH is adjusted to approximately between pH =6 to 8, or about 7.2, with a base, e.g., 1N sodium hydroxide. This solution is then sterile filtered through a 0.22 μm filter under aseptic conditions, filled into vials to the final desired volume, and subsequently capped with a rubber stopper and an over seal, such as an aluminum seal.

### Example 1. Antiviral and Anticoagulation Activities

This example tested the antiviral and anticoagulation activities of a bovine intestine heparin sample (HEP001) which had about 20% reduction of 6-O desulfation as compared to the conventional unfractionated heparin. Suitable bovine intestine heparin as tested herein is commercially sourced from South America. Enoxaparin (SelleckChem) and unfractionated heparin (UFH) (unfractionated porcine intestine heparin from Sigma) were used as control.

A Vero 76 cell line, comprised of kidney cells expressing ACE2, was used as the target cell, which was incubated with SARS-CoV live virus, in the presence/absence of a testing drug. EC₅₀ was measured by cytopathic effect (CPE). The results are listed below, along with each testing drug's anticoagulation activity as measured with conventional means.

| | EC₅₀ | EC₅₀ | Anticoagulation | Anti-factor |
|---|---|---|---|---|
| Drug | (μg/mL) | (μM) | (Xa U/mg) | IIa (U/mg) |
| HEP001 | 3.2 | 0.2 | 100* | 94.5 |
| Enoxaparin | >100 | N/A | 90-120 | NT |
| Heparin | 7.9 | 0.5 | 217 | NT |
| (UFH) | | | | |

*Rounded value. Actual value = 96.2 (Ratio of Anti Xa/IIa for HEP001 = 1)
NT = Not Tested
Ratio of Anti Xa/IIa for heparin (UFH) estimated at approximately 1.

The results, therefore, demonstrate that HEP001has sub-micromolar antiviral potency while enoxaparin did not exhibit anti-viral activity.

Unfractionated heparin (UFH) had similar antiviral activities as HEP001, but considerably higher (>2X) anticoagulant activities.

### Example 2. Clinical Trial for Treating SARS-CoV-2 Infections

A clinical trial will be run to test the efficacy of HEP001 in treating SARS-CoV-2 Infections. Patients will be recruited according to the following inclusion/exclusion criteria.

Inclusion Criteria
Subject (or legally authorized representative) provides written informed consent prior to initiation of any study procedures.
Understands and agrees to comply with planned study procedures.
Agrees to the collection of OP swabs and venous blood per protocol.
Male or non-pregnant female adult ≥18 years of age at time of enrollment.
Has laboratory-confirmed SARS-CoV-2 infection as determined by PCR, or other commercial or public health assay in any specimen <72 hours prior to randomization.
Illness of any duration, and at least one of the following:
   Radiographic infiltrates by imaging (chest X-ray, CT scan, etc.), or
   Clinical assessment (evidence of rales/crackles on exam) AND SpO2≤94% on room air, or
   Requiring mechanical ventilation and/or supplemental oxygen).
Women of childbearing potential must agree to use at least one primary form of contraception for the duration of the study (acceptable methods will be determined by the site).

Exclusion Criteria
ALT/AST >5 times the upper limit of normal.
Stage 4 severe chronic kidney disease or requiring dialysis (i.e. eGFR <30)
Pregnancy or breast feeding
Anticipated transfer to another hospital which is not a study site within 72 hours.
Known allergy to heparin products
Previous history of heparin-induced-thrombocytopenia (HIT)
Bleeding Risk:
   Clinical: Active bleeding; head trauma; intracranial surgery or stroke within 3 months; history of intracerebral arteriovenous malformation, cerebral aneurysm or mass lesions of the central nervous system; history of a bleeding diatheses; gastrointestinal bleeding within 6 weeks; presence of an epidural or spinal catheter; selected cases of recent surgery where IV therapeutic UFH is considered contraindicated
   Laboratory: Platelet count <50×10⁹/L, INR >2.0, or baseline aPTT >50 seconds prior to enrolment

The patients will be given 400 mg (subcutaneous) HEP001 daily for a period of 4, 7, 14, 21, or 28 days, or placebo. Other doses, including 100 mg, 200 mg, 600 mg, and 800 mg will also be tested.

Alternatively, patients will receive HEP001 via IV, such as at 18 U/kg/hr over a 24 hr period for up to 10 days, or placebo.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A method for treating an infection by a coronavirus in a subject in need thereof, comprising administering to the subject an effective amount of a bovine heparin having reduced sulfation as compared to unfractionated porcine intestine heparin (UPIH).

2. The method of claim 1, wherein the heparin is not chemically modified.

3. The method of claim 1 or 2, wherein the heparin has from 5% to 50% reduction of sulfation as compared to the UPIH.

4. The method of any one of claims 1-3, wherein the heparin has a reduced percentage of disaccharide units having trisulfation.

5. The method of claim 4, wherein the trisulfation is ΔUA2S-GlcNS6S.

6. The method of claim 5, wherein the heparin has from 8 to 25% reduction of ΔUA2S-GlcNS6S disaccharide units as compared to the UPIH.

7. The method of claim 5, wherein from 45% w/w to 60% w/w of the disaccharide units in the heparin have the trisulfation.

8. The method of any one of claims 1-3, wherein the heparin has a reduced percentage of disaccharide units having 6-O-sulfation.

9. The method of claim 8, wherein the heparin has from 5 to 50% reduction of 6-O-sulfated disaccharide units as compared to the UPIH.

10. The method of claim 8, wherein from 35% w/w to 80% w/w of the disaccharide units in the heparin have the 6-O-sulfation.

11. A method for treating an infection by a coronavirus in a subject in need thereof, comprising administering to the subject an effective amount of a bovine heparin having from 10% to 70% reduction of anti-factor IIa (Ma) or anti-factor Xa (fXa) activity as compared to unfractionated porcine intestine heparin (UPIH).

12. The method of claim 6, wherein the heparin has an anti-fIIa activity of 70 U/mg to 100 U/mg.

13. The method of any one of claims 1-12, wherein the heparin is unfractionated bovine intestine heparin.

14. The method of any one of claims 1-13, wherein the heparin is a non-chemically modified unfractionated bovine intestine heparin .

15. The method of any one of claims 1-14, wherein the coronavirus is selected from the group consisting of SARS-CoV, SARS-CoV-2 and MERS-CoV.

16. The method of claim 15, wherein the coronavirus is SARS-CoV-2.

17. The method of claim 16, wherein the subject suffers from at least a symptom of COVID-19.

18. The method of claim 16, wherein the subject is hospitalized.

19. The method of claim 16, wherein the subject is treated with mechanical ventilation.

20. The method of any one of claims 1-19, wherein heparin is administered at from 5 U/Kg/hour to 50 U/Kg/hour, followed by continuous infusion.

21. The method of any one of claims 1-19, wherein the heparin is administered at about 400 mg daily.

22. The method of any one of claims 1-21, wherein the subject is on anticoagulant treatment.

23. The method of any one of claims 1-22, further comprising administering to the subject an agent selected from the group consisting of lopinavir, ritonavir, oseltamivir, favipiravir, fingolimod, methylprednisolone, bevacizumab, chloroquine phosphate, chloroquine, hydroxychloroquine sulfate, remdesivir and combinations thereof.

24. The method of any one of claims 1-23, further comprising administering to the subject an inhibitor to a cytokine selected from the group consisting of IL-2, CD28, GM-CSF, INFγ, IL-1β, IL-18, IL-33, IL-6, and TNF.

25. A sterilized pharmaceutical composition comprising unfractionated bovine intestinal heparin and a pharmaceutically acceptable excipient, having a pH of about 7.2 in buffered saline solution, wherein the unfractionated bovine intestinal heparin has an anti-factor IIa activity of less than 100 U/mg as measured by USP methods.

26. The sterilized pharmaceutical composition of claim 25, wherein the sterilized pharmaceutical composition is formulated for inhalation, continuous infusion, intravenous infusion, or subcutaneous administration.

27. A sterilized pharmaceutical composition for administration with a nebulizer comprising unfractionated bovine intestinal heparin and a pharmaceutically acceptable excipient, wherein the unfractionated bovine intestinal heparin has an anti-factor IIa activity of less than 100 U/mg as measured by USP methods.

28. The sterilized pharmaceutical composition of claim 27, wherein the sterilized pharmaceutical composition is an aqueous composition.

29. The sterilized pharmaceutical composition of claim 27 or 28, wherein the sterilized pharmaceutical composition comprises a pharmaceutically acceptable excipient selected from a buffer, preservative, co-solvent, suspending agent, surfactant, tonicity adjusting agent, humectants, or combination thereof.

30. A sterilized pharmaceutical composition comprising unfractionated bovine intestinal heparin and a pharmaceutically acceptable excipient, wherein the sterilized pharmaceutical composition is a dry powder formulation, and further wherein the unfractionated bovine intestinal heparin has an anti-factor IIa activity of less than 100 U/mg as measured by USP methods.

31. The sterilized pharmaceutical composition of claim 30, wherein the sterilized pharmaceutical composition is a dry powder for inhalation.

32. The sterilized pharmaceutical composition of any one of claims 25-31, wherein the unfractionated bovine intestinal heparin is not chemically modified.

33. The sterilized pharmaceutical composition of any one of claims 25-32, wherein the sterilized pharmaceutical composition comprises unfractionated bovine intestinal heparin having an average molecular weight of from 15 KDa to 20 KDa, or from 16 KDa to 17 KDa, or about 16 KDa.

34. The sterilized pharmaceutical composition of any one of claims 25-33, wherein the unfractionated bovine intestinal heparin has a reduced percentage of N,3,6-trisulfated α-D-glucosamine (α-GlcN,3,6-triS) as compared to unfractionated porcine intestinal heparin.

35. The sterilized pharmaceutical composition of any one of claims 25-34, wherein the unfractionated bovine intestinal heparin has about 20% reduction of 6-O desulfation as compared to unfractionated porcine intestinal heparin.

36. The sterilized pharmaceutical composition of any one of claims 25-35, wherein the unfractionated bovine intestinal heparin is characterized by having one or more of:
less than 47% N,6-disulfated α-D-glucosamine (α-GlcN,6-diS) attached to 2-sulfated α-iduronic acid (α-IdA2S) as determined by NMR; or less than 2% N,3,6-trisulfated α-D-glucosamine (α-GlcN,3,6-triS) as determined by NMR.

37. The sterilized pharmaceutical composition of any one of claims 25-36, wherein the unfractionated bovine intestinal heparin has an anti-factor IIa activity of from 70 to less than 100 U/mg.

38. The sterilized pharmaceutical composition of any one of claims 25-37, wherein the sterilized pharmaceutical composition is sterilized via filtration.

39. The sterilized pharmaceutical composition of any one of claims 25-38, wherein the sterilized pharmaceutical composition has an anti-factor Xa to anti-factor IIa ratio of from 0.9-1.1.

40. The sterilized pharmaceutical composition of any one of claims 25-39, wherein the sterilized pharmaceutical composition has a final concentration of heparin of from 10 mg/mL to 100 mg/mL, or greater than 50 mg/mL.
